(19) United States
(12) Patent Application Publication
(10) Pub. No.: US 2001/0056078 A1
(43) Pub. Date: Dec. 27, 2001

(54) Novel method for treating gut disorders using polyanionic polyglycosides

(75) Inventor: Stephen Dealler, Burnley (GB)

Correspondence Address: BEYER WEAVER & THOMAS LLP, P.O. BOX 778, BERKELEY, CA, 94704-0778, US

(73) Assignee: Norton Healthcare Limited

(21) Appl. No.: 09/782,684

(22) Filed: Feb. 12, 2001

(30) Foreign Application Priority Data
Feb. 11, 2000 (GB) 0003048.6

(51) Int. Cl. A61K 31/715

(52) U.S. Cl. 514/54; 514/62

(57) ABSTRACT

The present invention relates to a method for combatting gut disorders such as Crohn's disease and ulcerative colitis using polyanionic polyglycosides (eg polysulphonated polyglycosides).

The present invention relates to a method for combatting gut inflammation (eg inflammatory bowel disease) or abnormal gut permeability by administering polyanionic polyglycosides (eg polysulphonated polyglycosides).

Inflammatory bowel disease is a disorder characterised by a red and inflamed bowel. One particular form of inflammatory bowel disease effects the large bowel and is known as ulcerative colitis. A second (often indistinguishable) form of inflammatory bowel disease known as Crohn's disease may effect any part of the digestive tract and the full thickness of the bowel wall can become inflamed. Ulcerative colitis and Crohn's disease may be treated surgically, by administering steroids or by a controlled diet (or frequently a combination of these).

Autism is commonly associated with abdominal complaints (such as abdominal pain, nausea, retching, constipation, inflammatory bowel disease and malabsorption). Traditionally the abdominal complaints have been looked upon as being separate to or caused by the psychological symptoms (Wing, 1997, Autism, 1, 13 to 23) and for this reason have undergone little investigation. The recent increase in the number of cases of autism reported in the UK and the USA has suggested that some factor apart from a psychological one is involved in its pathogenesis. The presence of peptides with opioid properties in the diet and urine of autistic subjects are consistent with the hypothesis that the psychological syndrome could be the result of the uptake of neuroactive compounds from the gut (Reichelt et al, 1991, Brain Dysfunct., 4, 308-319 and Reichelt et al, 1994, Dev. Brain Dysfunct., 7, 71-85). The finding that the gut absorbs compounds that are not absorbed in matched controls has suggested that in autistic subjects, diet-derived opioids may reach the brain as a result of increased permeability of the gut (ie a “leaky gut”) in which the barrier between the gut lumen and the blood is inadequate. There is therefore a renewed interest in the causes and effects of abnormal gut permeability.

Macromolecules are able to pass from the gut lumen into the blood by traversing epithelial cells but cannot normally pass between cells due to the presence of tight junctions. Toxins and inflammation are two potential causes of the disruption of tight junctions. Once disrupted, the effect will tend to be potentiated by the enhanced uptake of further toxins and/or the inflammatory response resulting from the initial breach of the epithelial barrier.

The present invention is based on the recognition that certain polyglycosides lead to an improvement of the tight junctions found between gut epithelial cells. More particularly, such polyglycosides are thought to interact with heparan binding sites to prevent or inhibit inflammation of the gut. The beneficial effects of such polyglycoside compounds is thought to be closely associated with the involvement of the sulphate metabolism in the pathogenesis of the gut abnormality (eg the sulphation of macromolecules such as glysoaminoglycans).

Thus viewed from one aspect the present invention provides a method for combatting (eg preventing or treating) a gut disorder in a subject, said method comprising:

administering to the subject an effective amount of a polyglycoside (or a mixture of polyglycosides) or a salt thereof (eg a calcium, sodium, magnesium, potassium or ammonium salt thereof),

wherein the polyglycoside comprises a chain of glycosidic residues substituted regularly or irregularly with an anionic substituent.

Preferably the gut disorder is abnormal gut permeability (“leaky gut”).

Preferably the gut disorder is gut inflammation such as for example an inflammatory bowel disease. Particularly preferably the gut disorder is ulcerative colitis or Crohn's disease.

Preferably the polyglycoside is capable of entering the intercellular fluid.

Without wishing to be bound by any theoretical considerations, the polyanionic polyglycosides are thought to interact with cellular heparan binding sites which are found on proteins on the cell surface and as these proteins are taken into the cell to be either destroyed or recycled, any compound bound to the heparan binding site enters the cell and has an effect. For instance, specific cytotoxic cytokinins may cause apoptosis in epithelial cells and lead to severe ulceration and further inflammation of the gut surface. Polyanionic polyglycosides present in the intercellular fluid also bind with heparan binding sites (and some would displace the normal compounds such as heparin) on cytokinins and when they themselves are taken into the cells, they are destroyed with the cytokinin and no effect by these compounds is seen. As such, the presence of the polyanionic polyglycoside will prevent inflammation and damage caused by cellular chemicals.

In a preferred embodiment, the polyglycoside is capable of interacting with a cellular heparan binding site.

In a preferred embodiment, the polyglycoside is capable of restoring disrupted tight junctions between epithelial cells.

In a preferred embodiment, the chain of glycosidic residues comprises a glucosamine residue.

In a preferred embodiment, the chain of glycosidic residues comprises a iduronic acid residue.

In a preferred embodiment, the chain of glycosidic residues comprises a glucoronic acid residue.

In a particularly preferred embodiment, the chain of glycosidic residues comprises a glucosamine residue and either a glucoronic acid or an iduronic acid residue.

In a preferred embodiment, the chain of glycosidic residues comprises a xylose residue.

Where the glycosidic chain is substituted regularly, it may comprise a repeating glycosidic unit. Preferably the repeating glycosidic unit is a mono-, di-, tri- or polysaccharide unit.

The anionic substituent may be selected from the group consisting of sulphate (eg SO₃⁻ or HSO₃) and carboxylate (eg CO₂⁻ or HCO₂). In a preferred embodiment, the anionic substituent is sulphate. In this case, there may be one, two or three sulphate substituents per glycosidic residue.

The anionic substituent may be directly or indirectly ring-bound. For example, the anionic substituent may be bound to the ring by a bridging group such as for example an epoxy group, an optionally alkyl-substituted imino group or an alkoxo (eg —CH₂O—) group.

The polyglycoside may be a natural or synthetic polyglycoside and may be homogeneous or heterogeneous. The glycosidic chain may be linear or non-linear. Typically the molecular weight of the polyglycoside is in the range 1 kDa to 8 MDa.

Specific examples of polyglycosides include heparin and salts thereof (eg calcium, sodium, magnesium or potassium salts thereof, preferably calcium and sodium salts thereof), low molecular weight fragments of heparin (obtainable by chemical or enzymatic depolymerisation of heparin) and salts thereof (eg sodium and calcium salts thereof) and heparinoids and salts thereof. By heparinoids is meant heparin derivatives including naturally occurring or synthetic highly sulphated polysaccharides of similar structure. These include sulphated glucosaminoglycans, glycosaminoglycan polysulphates and sulphated mucopolysaccharides; heparan sulphate, dermatan sulphate, chondroitin 4-sulphate and chondroitin 6-sulphate (and salts (eg sodium salts) and mixtures thereof); pentosan polysulphate sodium, sodium apolate and sulodexide.

Further specific examples of polyglycosides include λ-carrageenan, κ-carrageenan and υ-carrageenan (and mixtures thereof), dextran sulphate and salts thereof (eg sodium and potassium salts thereof, preferably sodium salts thereof); sulphated polyhyaluronic acid; colominic acid sulphate; and taurine

Mixtures of these polyglycosides may also be used in accordance with the invention.

Preferred polyglycosides for use in accordance with the invention are carrageenans, dextran sulphate and salts thereof which are thought to beneficially displace heparin and heparan from binding with binding sites on inflammatory molecules. Particularly preferred is pentosan polysulphate sodium which beneficially displaces heparin from binding sites and is advantageously orally administrable and is absorbed to a low degree. Pentosan polysulphate sodium is available (for example) in capsule form commercially under the trade mark Elmiron (Norton Healthcare).

The polyglycoside may be formulated as desired with conventional buffers, emulsifiers, stabilisers, viscosity enhancers, inert ingredients (such as excipients), additives and flavourings.

The polyglycoside may be administered by any conventional route such as parenterally or enterally including orally, rectally or intraveneously. The preferred administration route for the chosen polyglycoside may be readily determined by the skilled person and oral administration is generally preferred.

The polyglycoside may be administered in any convenient form such as capsule or tablet (preferably enterically coated) or syrup. The actual administration form for the chosen polyglycoside may be readily determined by the skilled person.

In a first preferred embodiment, the polyglycoside is administered in a novel sustained release formulation (eg in capsule or tablet form), particularly preferably a sustained release formulation which is adapted to release the polyglycoside in the ileum or colon (thereby reducing the number of daily doses).

A sustained release formulation which releases the active ingredient over a period of time (preferably in the ileum or colon) and thereby reduces the number of daily doses is advantageous. Such a sustained release formulation may use any conventional sustained release component such as (for example) a barrier, coating or erodable matrix.

In a second preferred embodiment, the polyglycoside is administered in a novel enterically coated unit (eg a unit in the form of an enterically coated capsule or tablet or in the form of enterically coated beads, pellets or granules contained in (for example) a tablet or capsule).

Viewed from a further aspect the present invention provides an orally administrable, sustained release formulation comprising a polyglycoside (or a mixture of polyglycosides) or a salt thereof (eg a calcium, sodium, magnesium, potassium or ammonium salt thereof) as hereinbefore defined.

Viewed from a yet further aspect the present invention provides an orally administrable formulation comprising a polyglycoside (or a mixture of polyglycosides) or a salt thereof (eg a calcium, sodium, magnesium, potassium or ammonium salt thereof) as hereinbefore defined in the form of an enterically coated unit (eg a unit in the form of an enterically coated capsule or tablet or in the form of enterically coated beads, pellets or granules contained in (for example) a tablet or capsule).

The polyglycoside may be administered in a dose which depends on the weight of the subject. This is readily determined by a person skilled in the art for the chosen polyglycoside. Typically the daily dose for a child under three years would be 50 mg orally and for a child between three and five years would be 100 mg orally. For a subject from the age of 6 years upwards the dose would typically be 100 mg twice daily. If diarrhoea results, the dose should be reduced.

Typically the selected dose is administered three times daily (eg orally) and the blood should be monitored to ensure that large amounts are not being absorbed after 48 hours. The administration regime will typically continue for several months (eg three to six months or longer) before any long term benefit is observed.

Viewed from a still further aspect the present invention provides an orally administrable formulation comprising a polyglycoside (or a mixture of polyglycosides) or a salt thereof (eg a calcium, sodium, magnesium, potassium or ammonium salt thereof) as hereinbefore defined, together with one or more carriers or excipients, wherein the polyglycoside is capable of entering the intercellular fluid and is present in an amount sufficient to prevent or inhibit a gut disorder (eg abnormal gut permeability or gut inflammation).

1. A method for combatting a gut disorder in a subject, said method comprising:
administering to the subject an effective amount of a polyglycoside (or a mixture of polyglycosides) or a salt thereof (eg a calcium, sodium, magnesium, potassium or ammonium salt thereof),
wherein the polyglycoside comprises a chain of glycosidic residues substituted regularly or irregularly with an anionic substituent.

2. The method as claimed in claim 1 wherein the gut disorder is gut inflammation.

3. The method as claimed in claim 1 wherein the gut disorder is inflammatory bowel disease.

4. The method as claimed in claim 1 wherein the gut disorder is ulcerative colitis or Crohn's disease.

5. The method as claimed in claim 1 wherein the gut disorder is abnormal gut: permeability.

6. The method as claimed in claim 1 wherein the polyglycoside salt is a calcium, sodium, magnesium, potassium or ammonium salt thereof.

7. The method as claimed in claim 1 wherein the polyglycoside is capable of interacting with a cellular heparan binding site.

8. The method as claimed in claim 1 wherein the chain of glycosidic residues comprises a glucosamine residue.

9. The method as claimed in claim 1 wherein the chain of glycosidic residues comprises an iduronic acid residue.

10. The method as claimed in claim 1 wherein the chain of glycosidic residues comprises a glucoronic acid residue.

11. The method as claimed in claim 1 wherein the chain of glycosidic residues comprises a glucosamine residue and either a glucoronic acid or an iduronic acid residue.

12. The method as claimed in claim 1 wherein the chain of glycosidic residues comprises a xylose residue.

13. The method as claimed in claim 1 wherein the glycosidic chain is substituted substantially regularly and comprises a repeating glycosidic unit being a mono-, di-, tri- or polysaccharide unit.

14. The method as claimed in claim 1 wherein the anionic substituent is selected from the group consisting of sulphate and carboxylate.

15. The method as claimed in claim 14 wherein the anionic substituent is sulphate.

16. The method as claimed in claim 15 wherein the glycosidic chain comprises one, two or three sulphate substituents per glycosidic residue.

17. The method as claimed in claim 1 wherein the anionic substituent is ring-bound by a bridging group.

18. The method as claimed in claim 17 wherein the bridging group is an epoxy group, an optionally alkyl-substituted imino group or an alkoxo group.

19. The method as claimed in claim 18 wherein the bridging group is an epoxy group.

20. The method as claimed in claim 1 wherein the polyglycoside is selected from the group consisting of heparin and salts thereof, low molecular weight fragments of heparin and salts thereof and heparinoids and salts thereof.

21. The method as claimed in claim 20 wherein the heparinoid is selected from the group consisting of sulphated glucosaminoglycans, glycosaminoglycan polysulphates, sulphated mucopolysaccharides, heparan sulphate, dermatan sulphate, chondroitin 4-sulphate, chondroitin 6-sulphate, pentosan polysulphate sodium, sodium apolate and sulodexide.

22. The method as claimed in claim 1 wherein the polyglycoside is selected from the group consisting of λ-carrageenan, κ-carrageenan, τ-carrageenan (and mixtures thereof), dextran sulphate and salts thereof, sulphated polyhyaluronic acid, colominic acid sulphate and taurine.

23. The method as claimed in claim 20 wherein the salt is a calcium or sodium salt.

24. The method as claimed in claim 22 wherein the polyglycoside is selected from the group consisting of λ-carrageenan, κ-carrageenan, τ-carrageenan and dextran sulphate and salts thereof.

25. The method as claimed in claim 20 wherein the polyglycoside is pentosan polysulphate sodium.

26. The method as claimed in claim 1 wherein the polyglycoside is in an orally administrable formulation.

27. The method as claimed in claim 26 wherein the orally administrable formulation is a sustained release formulation.

28. The method as claimed in claim 27 wherein the sustained release formulation is adapted to release the polyglycoside in the ileum or colon.

29. The method as claimed in claim 26 wherein the orally administrable formulation is in an enterically coated unit.

30. The method as claimed in claim 29 wherein the enterically coated unit is in the form of an enterically coated capsule or tablet or in the form of enterically coated beads, pellets or granules contained in a tablet or capsule.

31. An orally administrable, sustained release formulation comprising a polyglycoside (or a mixture of polyglycosides) or a salt thereof, wherein the polyglycoside comprises a chain of glycosidic residues substituted regularly or irregularly with an anionic substituent.

32. An orally administrable formulation in the form of an enterically coated unit comprising a polyglycoside (or a mixture of polyglycosides) or a salt thereof, wherein the polyglycoside comprises a chain of glycosidic residues substituted regularly or irregularly with an anionic substituent.

33. An orally administrable formulation as claimed in claim 32 wherein the enterically coated unit is an enterically coated capsule or tablet or enterically coated beads, pellets or granules contained in a tablet or capsule.

34. An orally administrable formulation comprising a polyglycoside or a salt thereof, together with one or more carriers or excipients, wherein the polyglycoside comprises a chain of glycosidic residues substituted regularly or irregularly with an anionic substituent and wherein the polyglycoside is capable of entering the intercellular fluid and is present in an amount sufficient to prevent or inhibit a gut disorder.

35. A oral administrable formulation as claimed in claim 34 wherein the gut disorder is gut inflammation or abnormal gut permeability.
